(19) United States
(12) Patent Application Publication
(10) Pub. No.: US 2024/0360511 A1
(43) Pub. Date: Oct. 31, 2024

(54) METHOD FOR IDENTIFYING AND DIAGNOSING GENETIC DISORDERS AND SYETEM THEREOF

(71) Applicant: CARDIAI TECHNOLOGIES, Calgary (CA)

(72) Inventors: MINAL BORKAR TRIPATHI, Calgary (CA); Sidharth Singh Bhinder, Calgary (CA); Anmol Singh Kapoor, Calgary (CA); Amrita Kaur, Calgary (CA)

(21) Appl. No.: 18/647,020

(22) Filed: Apr. 26, 2024

Related U.S. Application Data
(60) Provisional application No. 63/498,631, filed on Apr. 27, 2023.

(51) Int. Cl. C12Q 1/6883; G16B 20/00; G16H 10/20; G16H 50/20

(52) U.S. Cl. CPC C12Q 1/6883; G16B 20/00; G16H 10/20; G16H 50/20; C12Q 2600/156

(57) ABSTRACT

The present disclosure relates to a method for identifying and diagnosing genetic disorders. The method comprises obtaining a sample from a subject. The method also comprises extracting deoxyribonucleic acid (DNA) from the sample collected. The method also comprises storing properly the extracted deoxyribonucleic acid (DNA) from the collected sample. The method also comprises analyzing for the presence of a genetic mutation or a genetic profile associated with the disorder. The method also comprises comparing the identified mutation or profile with a database of known mutations or profiles associated with various genetic disorders. The method also comprises providing probable diagnosis of the disorder based on the comparative results obtained.

### CROSS-REFERENCE TO RELATED APPLICATIONS

This application claims the benefit of Indian Application No. 63/498,631 titled “METHODS AND SYSTEMS FOR IDENTIFYING AND DIAGNOSING GENETIC DISORDERS” filed by the applicant on Apr. 27, 2023, which is incorporated herein by reference in its entirety.

### FIELD OF THE INVENTION

Embodiments of the present invention relate to the field of E profiling and more particularly, relate to a method for identifying and diagnosing genetic disorders and a system thereof.

### DESCRIPTION OF THE RELATED ART

Genetic disorders are a diverse group of disorders that are caused by changes or mutations in an individual's DNA. These mutations can be inherited from one or both parents or can arise spontaneously during the individual's lifetime. Genetic disorders can affect any part of the body and can have a wide range of symptoms and effects, ranging from mild to severe and life-threatening.

There are thousands of known genetic disorders, each with its unique set of symptoms and genetic mutations. Examples of genetic disorders include cystic fibrosis, sickle cell anemia, Huntington's disease, and Down syndrome. Some genetic disorders are caused by mutations in a single gene, while others are caused by a combination of genetic and environmental factors.

Identifying and diagnosing genetic disorders is important for providing appropriate treatment and management of the disorder. Genetic testing, including DNA sequencing, is a valuable tool for diagnosing genetic disorders. Genetic testing can help identify the specific genetic mutation causing the disorder and can help guide treatment and management strategies. Additionally, genetic testing can be used to identify carriers of genetic mutations, which can be useful in family planning and genetic counseling.

Overall, genetic disorders can have a significant impact on an individual's health and quality of life. The ability to identify and diagnose genetic disorders through genetic testing is critical for ensuring appropriate treatment and management of these disorders. However, there are many challenges and limitations associated with genetic disorder identification and diagnosis, which are crucial in understanding and treating various medical conditions.

As many genetic disorders are caused by mutations in multiple genes or by complex interactions between genes and the environment. This complexity makes it challenging to pinpoint the exact genetic cause using traditional methods. Further, the field of genetic studies is still evolving and current knowledge about human genome is still limited. There are parts of the genome that are poorly understood or have unknown functions, that makes it difficult to interpret many genetic variations.

Additionally, even the genetic mutations can vary significantly between individuals, even those with the same disorder. This variability makes it difficult to establish clear correlations and may result in missed diagnoses. Many of the existing genetic testing methods have limited ability to detect all relevant mutations and limited ability to accurately identify mutations without false positives.

Furthermore, many of the currently available advanced genetic testing methods are expensive and not readily accessible to all patients. In addition to this, another set of challenges are associated with the genetic variant identification itself, which require expertise in differentiating between variants, as not all the variants are associated with diseases or genetic disorders.

Accordingly, to overcome the disadvantages of the prior art, there is an urgent need for a technical solution that overcomes the above-stated limitations in the prior arts. The present invention provides a method and a system for identifying and diagnosing genetic disorder.

### SUMMARY OF THE INVENTION

The present disclosure solves all the above major limitations of a method and system for identifying and diagnosing genetic disorders. Further, the present disclosure ensures that the disclosed invention may fulfil following objectives.

An objective of the present disclosure is to develop an effective and reliable method for identifying and diagnosing genetic disorders.

Another objective of the present disclosure is to ensure high accuracy in identifying genetic and to minimize false positives and negatives, providing reliable diagnostic information to healthcare providers and patients.

Another objective of the present disclosure is to detect a wide range of genetic variations to capture the full spectrum of genetic disorders.

Another objective of the present disclosure is to ensure scalability for being applied to large-scale screenings as well as individual patient diagnosis, facilitating early detection and intervention for managing genetic disorders.

Another objective of the present disclosure is to ensure easy accessibility for all, particularly for those in resource-limited settings.

Another objective of the present disclosure is to ensure that the proposed system may easily integrate with existing genetic testing and diagnostic methods seamlessly into clinical care pathways, ensuring that healthcare providers can easily access and interpret genetic information to inform patient management and treatment options.

Yet another objective of the present disclosure is to develop a less complex method for identifying genetic disorders.

Yet another objective of the present disclosure is to develop a cost-effective method for identifying genetic disorders.

Yet another objective of the present disclosure is to develop a resource-efficient method for identifying genetic disorder.

Yet another objective of the present disclosure is to develop a method for identifying genetic variants that has high sensitivity and specificity.

Embodiments of the present invention relate to a method for identifying and diagnosing genetic disorders. The method includes obtaining a sample from a subject. The method also includes extracting deoxyribonucleic acid (DNA) from the sample collected. The method also includes storing properly the extracted deoxyribonucleic acid (DNA) from the collected sample. The method also includes analyzing for the presence of a genetic mutation or a genetic profile associated with the disorder. The method also includes comparing the identified mutation or profile with a database of known mutations or profiles associated with various genetic disorders. The method also includes providing probable diagnosis of the disorder based on the comparative results obtained.

In accordance with an embodiment of the present invention, the analysis is performed using various techniques, including deoxyribonucleic acid (DNA) sequencing, polymerase chain reaction (PCR), and/or fluorescence in situ hybridization (FISH).

In accordance with an embodiment of the present invention, the database is created using information from previous research studies or clinical trials.

Embodiments of the present invention relate to a computer implemented system of identifying and diagnosing genetic disorders. The system comprising a collection and extraction unit configured to obtain a sample from a subject and extract and store deoxyribonucleic acid (DNA) from the sample collected. The system also comprising a processing and analytics assembly linked to the collection and extraction unit and the processing and analytics assembly further comprises a comprehensive genetic analysis unit, a functional genomics analysis unit, and a comparative analysis unit. The system also comprising a database unit linked to the processing and analytics assembly and the database unit configured for easy storage of information from previous research studies or clinical trials and easy retrieval of information from previous research studies or clinical trials. The system also comprising a smart diagnostic unit linked to the processing and analytics assembly and the smart diagnostic unit configured to generate probable diagnosis based on the results obtained from the comparative analysis unit. The system also comprising a network unit linking the collection and extraction unit to the processing and analytics assembly and the network unit capable of connecting to external computing resources.

In accordance with an embodiment of the present invention, the comprehensive genetic analysis unit configured to provide a comprehensive analysis of genetic mutations associated with various disorders.

In accordance with an embodiment of the present invention, the functional genomics analysis unit assess the functional consequences of genetic mutations identified by the comprehensive genetic analysis unit on a plurality of factors, including gene expression, protein function, and/or cellular pathways.

In accordance with an embodiment of the present invention, the comparative analysis unit compares the identified mutation or profile with the known mutations or profiles associated with various genetic disorders retrieved form the database unit.

In accordance with an embodiment of the present invention, the comparative analysis unit identifies, records, and reports matches or similarities between the identified mutation or profile with the known mutations and profiles associated with various genetic disorders retrieved form the database unit.

In accordance with an embodiment of the present invention, the smart diagnostic unit scores, ranks and interprets the matched data obtained from the processing and analytics assembly.

In accordance with an embodiment of the present invention, the system also includes a user interface linked or integrated with the smart diagnostic unit and the user interface configured to serve as an interactive graphical user interface.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 is a flowchart illustrating a method for identifying and diagnosing genetic disorders, in accordance with one embodiment of the present invention; and

FIG. 2 is a block diagram illustrating a computer implemented system of identifying and diagnosing genetic disorders, in accordance with an embodiment of the present invention.

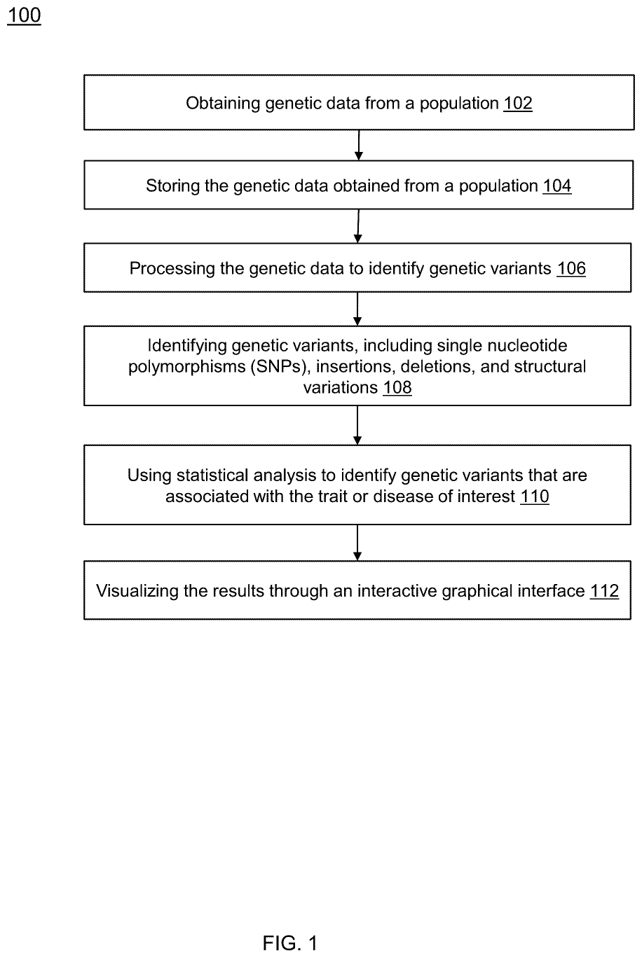

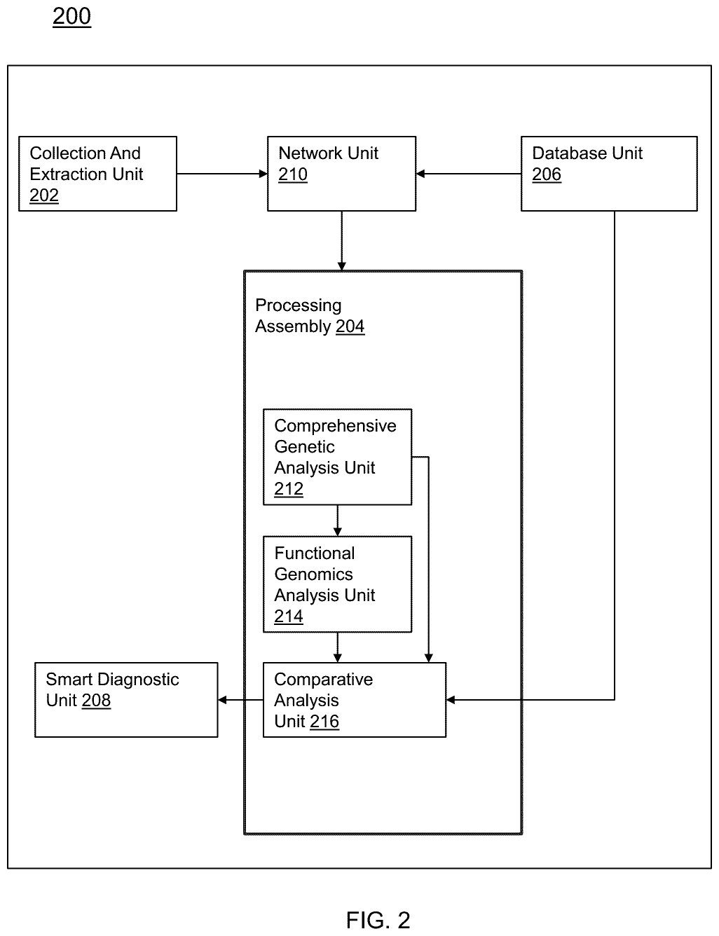

The FIGS. 1 and 2 are illustrated in the accompanying drawings, which like reference letters indicate corresponding parts in the various figures. It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

The principles of the present invention and their advantages are best understood by referring to FIGS. 1 and 2. In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The following brief definition of terms shall apply throughout the present invention:

FIG. 1 is a flowchart illustrating a method 100 for identifying and diagnosing genetic disorders, in accordance with one embodiment of the present invention. The method 100 may comprise the following steps.

At 102, obtaining a sample from a subject.

In a preferred embodiment, the sample collected from a subject, which may be blood, saliva, a tissue sample and/or other bodily fluids. In an embodiment of the present disclosure, the subject may be any living or dead organism or life form, such as, humans, animals, and so.

At 104, extracting deoxyribonucleic acid (DNA) from the sample collected.

At 106, storing properly the extracted deoxyribonucleic acid (DNA) from the collected sample.

At 108, analyzing for the presence of a genetic mutation or a genetic profile associated with the disorder.

In a preferred embodiment, various techniques may be used to analyze the sample, including, but not limited to, sequencing, genotyping, and other genetic analysis techniques.

At 110, comparing the identified mutation or profile with a database of known mutations or profiles associated with various genetic disorders.

At 112, providing probable diagnosis of the disorder based on the comparative results obtained.

The analysis may be performed using various techniques, including deoxyribonucleic acid (DNA) sequencing, polymerase chain reaction (PCR), and/or fluorescence in situ hybridization (FISH).

The database may be created using information from previous research studies or clinical trials.

In an embodiment of the present disclosure, the extraction of deoxyribonucleic acid (DNA) from the collected sample may involves breaking open cells to release the deoxyribonucleic acid (DNA), removing proteins and other contaminants, and isolating the deoxyribonucleic acid (DNA) using techniques such as, but not limited to, phenol-chloroform extraction, silica membrane-based purification, and/or magnetic bead-based purification.

In an embodiment of the present disclosure, the proper storage of the extracted deoxyribonucleic acid (DNA) maintains its integrity and stability. The deoxyribonucleic acid (DNA) samples extracted may be stored in specialized storage solutions, such as deoxyribonucleic acid (DNA) stabilizing buffers or Tris-EDTA (TE) buffer, at specific temperature to prevent degradation over time.

In an embodiment of the present disclosure, after extraction the genetic material may be sequenced to determine the order of nucleotides in the DNA molecule. This sequencing data may be generated in the form of raw sequencing reads or processed into assembled genomes or genetic variants. The sequencing data may be converted into a digital format that can be easily stored and accessed online. This digitization process involves encoding the genetic information into a standardized format, such as FASTQ, BAM, VCF, and/or FASTA.

In some embodiments, the digitized genetic data is uploaded to secure cloud-based storage platforms designed specifically for genetic data storage offering scalable storage, data encryption, access controls, and redundancy to ensure the security and integrity of the stored data. Relevant metadata, such as sample identifiers, experimental protocols, quality metrics, and clinical annotations, may also be stored online along with the genetic data stored.

In an embodiment of the present disclosure, the analysis of extracted and stored DNA data may include pre-processing of the data including cleaning and preparing the data for analysis. The pre-processing may also involve quality control checks, data normalization, and filtering to remove low-quality reads or artifacts that could affect the accuracy of the analysis.

In an embodiment of the present disclosure, the analysis of extracted and stored DNA data may include using a plurality of bioinformatics tools and algorithms to identify differences in the DNA sequence compared to a reference genome. In some embodiment, the genetic variants are annotated with information about their genomic coordinates, functional consequences, allele frequencies, and known associations with genetic disorders. In some embodiments, bioinformatics databases and tools, such as ClinVar, OMIM, and population databases, may be used for analysis.

In some embodiments, computational prediction tools are used to evaluate the clinical significance of the genetic variants and their relevance to the associated genetic disorders. In some embodiments, the databases may be curated databases of disease-causing mutations, genotype-phenotype associations, or clinical guidelines. In some embodiments, the identified genetic mutations or profiles may be compared with known mutations or profiles associated with specific disorders, to determine the likelihood of the observed variants contributing to the disorder.

In an alternate embodiment, for comparison of genetic data various bioinformatics tools and databases may be used, such as, Variant Annotation and Interpretation Tools including, Variant Effect Predictor, and SnpEff, Variant Calling and Comparison Tools including, Genome Analysis Toolkit and Samtools, Variant Frequency Databases including, gnomAD and Exome Aggregation Consortium, Clinical Variant Interpretation Tools, including, ClinVar and InterVar, Genotype-Phenotype Association Tools including, Phenolyzer and Disease Variant Store, Population Genetics Tools including, PLINK and ADMIXTURE, and Phylogenetic Analysis Tools including RAxML and Bayesian Evolutionary Analysis Sampling Trees.

FIG. 2 is a block diagram illustrating a computer implemented system 200 of identifying and diagnosing genetic disorders, in accordance with an embodiment of the present invention.

The system 200 may comprise a collection and extraction unit 202, a processing and analytics assembly 204, a database unit 206, a smart diagnostic unit 208, and a network unit 210.

The collection and extraction unit 202 may be configured to obtain a sample from a subject and extract and store deoxyribonucleic acid (DNA) from the sample collected.

In a preferred embodiment, the deoxyribonucleic acid (DNA) may be extracted from the biological samples collected in form of blood, saliva, tissue, and/or other bodily fluids. Proper handling of biological sample is performed to preserve of the sample as well as extracted deoxyribonucleic acid (DNA), as per the standard protocols established.

The processing and analytics assembly 204 may be linked to the collection and extraction unit 202 and the processing and analytics assembly 204 may further comprise a comprehensive genetic analysis unit 212, a functional genomics analysis unit 214, and a comparative analysis unit 216.

In some embodiments, prior to the processing and analytics assembly 204 the system 200 may have a data pre-processing unit and a data storage unit data prior. The data preprocessing unit may perform quality control checks to filter out low-quality reads, and sequencing artifacts, and/or adapter sequences, sort out reads based on quality scores, length, or other pre-defined criteria, perform read alignment as per a reference genome. In some embodiments, the data storage unit may be a secure local or a remote storage module configured to easy store and retrieve a large dataset.

The database unit 206 linked to the processing and analytics assembly 204 and the database unit 206 configured for: easy storage of information from previous research studies or clinical trials, and easy retrieval of information from previous research studies or clinical trials.

In an embodiment of the present disclosure, the database unit 206 may contain curated genetic information, including known mutations associated with specific genetic disorders, population-level allele frequencies, and functional annotations.

The smart diagnostic unit 208 linked to the processing and analytics assembly 204 and the smart diagnostic unit 208 configured to generate probable diagnosis based on the results obtained from the comparative analysis unit 216.

The network unit 210 linking the collection and extraction unit to the processing and analytics assembly 204 and the network unit 210 capable of connecting to external computing resources.

In a preferred embodiment, the network unit 210 may link the processing and analytics assembly 204 to graphical interface via the smart diagnostic unit 208. The network unit 210 may enable the high-speed transfer of raw and processed genetic data.

In a preferred embodiment, the network unit 210 enables the transfer of raw genomic data including, but not limited to, genome sequences or genetic data repositories within the system 200. In a preferred embodiment, the network unit 210 inter-communication between various components of the system 200 including, facilitating high-speed data transfer, seamless data flow, secure data transmission, and information exchange.

In some embodiments, the network unit 210 may provide high-speed internet connectivity and may include a wireless communication network, a 3G communication network, a 4G communication network, a 5G communication network, or any combination thereof any transceiver, or any combination thereof. Embodiments of the present disclosure are intended to cover any communication network technology, including known, prior art or later developed technologies. In some embodiments, the network unit 210 may be a local area communication network (LAN), wide area communication network (WAN), private networks, and such or a combination thereof.

The comprehensive genetic analysis unit 212 may be configured to provide a comprehensive analysis of genetic mutations associated with various disorders.

In some embodiments, the comprehensive genetic analysis unit 212 may ensure that data obtained for the genetic analysis data and the genetic data stored in the database unit 206 are in compatible formats and adhere to standardized data formats and conventions. In a preferred embodiment, the comprehensive genetic analysis unit 212 may involve preprocessing and normalizing the data to ensure consistency and compatibility.

In some embodiments, the comprehensive genetic analysis unit 212 may integrate technologies such as, DNA sequencing, polymerase chain reaction (PCR), fluorescence in situ hybridization (FISH), and/or others to provide a comprehensive analysis of genetic mutations associated with various disorders.

The functional genomics analysis unit 214 may assess the functional consequences of genetic mutations identified by the comprehensive genetic analysis unit on a plurality of factors, including gene expression, protein function, and/or cellular pathways.

In some embodiments, the functional genomics analysis unit 214 may identify functional assays to assess the functional consequences of these mutations on gene expression, cellular pathways and so. The identification of functional assays may provide valuable insights into the pathogenicity of the identified mutations and can help correlate genetic variants with genetic disorders, improving the accuracy of diagnosis.

The comparative analysis unit 216 compares the identified mutation or profile with the known mutations or profiles associated with various genetic disorders retrieved form the database unit.

The comparative analysis unit 216 may identify, record, and report matches or similarities between the identified mutation or profile with the known mutations and profiles associated with various genetic disorders retrieved form the database unit 206.

In some embodiments, the comparative analysis unit 216 may use various bioinformatics tools and annotate the variants with information such as genomic coordinates, variant type such as, single nucleotide variant, insertion, deletion. In an embodiment of the present disclosure the comparative analysis unit 216 may perform database querying by using the annotated variants from the analysis data as search queries.

In an embodiment of the present disclosure, the comparative analysis unit 216 may compare the annotated variants obtained from the analysis data with the genetic data stored in the database unit 206 to identify matches or similarities. In an embodiment of the present disclosure, the comparative analysis unit 216 may apply filtering criteria to prioritize variants based on factors such as pathogenicity, allele frequency, and relevant phenotype.

The smart diagnostic unit 208 may score, rank and interpret the matched data obtained from the processing and analytics assembly.

In an embodiment of the present disclosure, the smart diagnostic unit 208 may employ machine learning and artificial intelligence algorithms, to intelligently interprets complex genomic data and identifies patterns or correlations between genetic variants and clinical outcomes. In an embodiment of the present disclosure, the smart diagnostic unit 208 may perform automated analysis, by using predictive modeling for genetic disorders.

In an embodiment of the present disclosure, the smart diagnostic unit 208 may score and rank the matched variants based on their potential relevance to the observed clinical phenotype, disease association, and predicted impact on gene function. In some embodiments, the smart diagnostic unit 208 may consider information such as gene expression data, functional assays, and such, coupled with the result obtained from the comparative analysis unit 216, to provide probable diagnosis.

The system 100 also includes a user interface linked or integrated with the smart diagnostic unit and the user interface configured to serve as an interactive graphical user interface.

In some embodiments, the smart diagnostic unit 208 may generate a comprehensive report summarizing the findings of the genetic analysis, including the identified variants, their clinical significance, supporting evidence, and recommendations for further evaluation or management. In an embodiment of the present disclosure, the results generated by the smart diagnostic unit 208 may be communicated to the user interface in a clear and actionable format.

In a preferred embodiment, the user interface may be any graphical user interface, including known, prior art, or later developed technologies. In some embodiments, the graphical user interface may have capacity to display reports and employ a plurality of visualization tools for interactive display of results obtained.

In a preferred embodiment, the disclosed method 100 and system 200 may be used to identify and diagnose a wide variety of genetic disorders including, but not limited to, inherited disorders, acquired disorders, and developmental disorders.

In an alternate embodiment, the processing and analytics assembly 204 may use next-generation sequencing technologies offer high-throughput sequencing capabilities, allowing for the simultaneous analysis of multiple genes or even the entire genome.

This approach can identify not only single nucleotide variants (SNVs) but also insertions, deletions, copy number variations (CNVs), and structural rearrangements associated with genetic disorders. Use of NSG may provide a comprehensive genetic profile and enable the detection of novel or rare disorders that might be missed by traditional diagnosing methods.

In an alternate embodiment, the processing and analytics assembly 204 may integrate genomic data, such as transcriptomics, proteomics, metabolomics, and epigenomics, to obtain more comprehensive understanding of the genetic disorders. The integration may enable the identification of disease pathogenesis, biomarker discovery, and personalized treatment strategies.

In some embodiments, the machine learning and artificial algorithms used for genetic data analysis may be support vector machines, random forests, neural networks such as convolutional neural networks (CNNs) and recurrent neural networks (RNNs), bayesian networks, hidden Markov models (HMMs), k-mer-based algorithms, clustering algorithms, dimensionality reduction techniques, and deep generative models. In an embodiment of the present disclosure, the suitable machine learning and artificial algorithms are into the processing and analytics assembly 204 and the smart diagnostic unit 206 of the system 200 to extract meaningful insights from complex genomic datasets, improve diagnostic accuracy, uncover genotype-phenotype associations, and understanding of the genetic basis of diseases.

In a best mode of operation, the method 100 involves obtaining a sample from a subject, which can be blood, saliva, or tissue sample, and analyzing it for the presence of a genetic mutation or a genetic profile associated with the disorder. The analysis may be performed using various techniques, including DNA sequencing, polymerase chain reaction (PCR), or fluorescence in situ hybridization (FISH). These techniques may help identify the specific mutation or genetic profile that is associated with the disorder.

Once the genetic mutation or profile is identified, the method 100 may provide a diagnosis of the disorder. The diagnosis may be made by comparing the identified mutation or profile with a database of known mutations or profiles associated with various genetic disorders. The database can be created using information from previous research studies or clinical trials. Once a diagnosis may be made, appropriate treatment options can be considered, which may include genetic counseling, medication, or gene therapy.

Thereby, the method 100 disclosed may be used to diagnose a wide range of genetic disorders, including inherited disorders such as cystic fibrosis, Huntington's disease, and sickle cell anemia, as well as acquired disorders such as certain types of cancer. The ability of the method 100 to accurately diagnose genetic disorders may be critical for providing patients with appropriate treatment and management of disorder. The disclosed method 100 for identifying and diagnosing a genetic disorder may be a crucial tool for healthcare professionals and researchers to accurately diagnose and treat patients with genetic disorders.

In conclusion, the disclosed invention relates to a method 100 and system 200 for identifying and diagnosing genetic disorders. The method 100 includes obtaining a sample from a subject. The sample can be obtained from a variety of sources, including but not limited to blood, tissue, saliva, or other body fluid or tissue sample. Once the sample has been obtained, the sample is analyzed to identify a genetic mutation or a genetic profile associated with the disorder. Various techniques may be used to analyze the sample, including but not limited to sequencing, genotyping, and other genetic analysis techniques. Once the mutation or genetic profile has been identified, a diagnosis is provided based on the identified mutation or genetic profile. The diagnosis can be provided directly to the patient, or can be provided to a medical professional or other healthcare provider for interpretation and treatment.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A method for identifying and diagnosing genetic disorders, the method comprising:
obtaining a sample from a subject;
extracting deoxyribonucleic acid (DNA) from the sample collected;
storing properly the extracted deoxyribonucleic acid (DNA) from the collected sample;
analyzing for the presence of a genetic mutation or a genetic profile associated with the disorder;
comparing the identified mutation or profile with a database of known mutations or profiles associated with various genetic disorders; and
providing probable diagnosis of the disorder based on the comparative results obtained.

2. The method of claim 1, wherein the analysis is performed using various techniques, including deoxyribonucleic acid (DNA) sequencing, polymerase chain reaction (PCR), and/or fluorescence in situ hybridization (FISH).

3. The method of claim 1, wherein the database is created using information from previous research studies or clinical trials.

4. A computer implemented system of identifying and diagnosing genetic disorders, the system comprising:
a collection and extraction unit configured to obtain a sample from a subject; and
extract and store deoxyribonucleic acid (DNA) from the sample collected;
a processing and analytics assembly linked to the collection and extraction unit, the processing and analytics assembly further comprises:
a comprehensive genetic analysis unit;
a functional genomics analysis unit; and
a comparative analysis unit;
a database unit linked to the processing and analytics assembly, the database unit configured for:
easy storage of information from previous research studies or clinical trials; and
easy retrieval of information from previous research studies or clinical trials;
a smart diagnostic unit linked to the processing and analytics assembly, the smart diagnostic unit configured to generate probable diagnosis based on the results obtained from the comparative analysis unit; and
a network unit linking the collection and extraction unit to the processing and analytics assembly, the network unit capable of connecting to external computing resources.

5. The system of claim 4, wherein the comprehensive genetic analysis unit configured to provide a comprehensive analysis of genetic mutations associated with various disorders.

6. The system of claim 4, wherein the functional genomics analysis unit assess the functional consequences of genetic mutations identified by the comprehensive genetic analysis unit on a plurality of factors, including gene expression, protein function, and/or cellular pathways.

7. The system of claim 4, wherein the comparative analysis unit compares the identified mutation or profile with the known mutations or profiles associated with various genetic disorders retrieved form the database unit.

8. The system of claim 7, wherein the comparative analysis unit identifies, records, and reports matches or similarities between the identified mutation or profile with the known mutations and profiles associated with various genetic disorders retrieved form the database unit.

9. The system of claim 4, wherein the smart diagnostic unit scores, ranks and interprets the matched data obtained from the processing and analytics assembly.

10. The system of claim 4, wherein the system also includes a user interface linked or integrated with the smart diagnostic unit and the user interface configured to serve as an interactive graphical user interface.
